(19) United States
(12) Patent Application Publication
(10) Pub. No.: US 2024/0065549 A1
(43) Pub. Date: Feb. 29, 2024

(54) TONOMETERS WITH SENSOR ARRAYS FOR CORNEAL PROFILE MEASUREMENT

(71) Applicant: Twenty Twenty Therapeutics LLC, South San Francisco, CA (US)

(72) Inventors: Supriyo Sinha, Menlo Park, CA (US); Hojr Sedaghat Pisheh, South San Francisco, CA (US); Dimitri Azar, South San Francisco, CA (US)

(21) Appl. No.: 18/456,086

(22) Filed: Aug. 25, 2023

Related U.S. Application Data
(60) Provisional application No. 63/401,966, filed on Aug. 29, 2022.

(51) Int. Cl. A61B 3/16; A61F 9/00

(52) U.S. Cl. CPC A61B 3/165; A61F 9/0008

(57) ABSTRACT

Systems, devices, and methods for determining an intraocular pressure (IOP) of an eye are provided. A system may include a pump configured to generate a puff of air and a nozzle configured to direct the puff of air along a first axis toward the eye. The system may further include a light source distal of the nozzle and directed to emit a beam of light toward the first linear optical sensor along a second axis transverse to the first axis. A first portion of the beam of light illuminates a lateral surface of the eye and a second portion of the beam of light passes in front of the eye. The system may further include a first linear optical sensor disposed distal of the nozzle and configured to receive the second portion of the beam of light.

### CROSS-REFERENCE TO RELATED APPLICATIONS

The present claims the benefit of and priority to U.S. Provisional Patent Application No. 63/401,966, filed Aug. 29, 2022, the entirety of which is incorporated by reference herein.

### TECHNICAL FIELD

The present disclosure relates generally to devices and methods for measuring intraocular pressure (IOP) in human eyes, and in particular to devices that generate an air puff for noncontact tonometry.

### BACKGROUND

Intraocular pressure (IOP) quantifies the pressure of the aqueous humor inside the eye. Many individuals suffer from disorders, such as glaucoma, that cause chronic heightened IOP. Over time, heightened IOP can cause damage to the optical nerve of the eye, leading to loss of vision. Effective treatment of glaucoma (e.g., using pharmaceutical agents) requires adherence to dosage schedules and a knowledge of the patient's IOP. The more current or recent the measurement is, the more relevant it will be and hence the more effective the resulting treatment can be. The IOP for a given patient can vary significantly based on the time of day, exercise, recency of medication use, and other factors. This means that any given measurement is subject to uncertainty, so it may take a plurality of measurements over time to provide confidence as to the health status of the patient. IOP measurements performed in a doctor's office typically only take place once or twice per year. These infrequent measurements are less capable to account for variation in patient IOP. Annual or biannual measurements in a doctor's office may also grow stale or obsolete due to time lag since the previous measurement. Frequent measurements at home could allow for better treatment at lower cost.

IOP may be measured through tonometry. However, some currently available tonometry devices and techniques and devices have numerous drawbacks. Contact tonometry is performed in a medical setting, and carries both an infection risk and a risk of injury. The procedure may also require numbing of the patient's eyes, resulting in both inconvenience and discomfort. Noncontact tonometry involves directing a puff or jet of air at the patient's eye and measuring the resulting deflection of the eye. The measurement of the deflection may be performed by optical components or sensors. In some aspects, some devices may require precise placement of the cornea in multiple axes to obtain an accurate corneal deflection measurement. Misalignment in one or more axes may result in an inaccurate or inconclusive measurement. For this reason, non-contact tonometry is often performed under the supervision or control of an optometrist or ophthalmologist instead of at the patient's home.

### SUMMARY

The present disclosure advantageously describes embodiments of non-contact tonometer devices including a light source and optical sensor or sensor array that may allow for some misalignment of the eye in one or more axes. The tonometers described herein include a light source configured to emit a beam of light transverse to an axis of the air stream of the tonometer device. The beam has a width or profile in the air jet axis. The optical sensor array includes a plurality of sensor elements positioned along a line or curve and positioned in an optical path of the light source. At least a portion of the light from the light source may be obscured by the profile of the user's cornea such that at least some of the sensor elements are prevented from receiving the light from the beam. Accordingly, the number of sensor elements that detect or register light from the light source may indicate or represent the corneal profile. A plurality of measurements may be obtained during the delivery of the air jet to determine the change in the shape (e.g., flattening) of the cornea over time. Based on these measurements and the pressure of the air jet, the IOP may be determined. In some aspects, the embodiments described herein may allow for more flexibility and misalignment in one or more axes. For example, the tonometry devices described herein may allow for IOP measurements to be obtained while the eye is positioned within a suitable range of positions in the air jet axis. The range of positions afforded by the embodiments of the present disclosure may make the tonometry devices more suitable for home use by the patients themselves.

According to one aspect of the present disclosure, a system is provided for determining an intraocular pressure (IOP) of an eye. The system may include: a pump configured to generate a puff of air. The system may further include a nozzle in communication with the pump and configured to direct the puff of air along a first axis toward the eye. The system may further include a light source disposed distal of the nozzle and directed to emit a beam of light toward the first linear optical sensor along a second axis transverse to the first axis, wherein the beam of light comprises a width in the first axis such that a first portion of the beam of light illuminates a lateral surface of the eye and a second portion of the beam of light passes in front of the eye. The system may further include a first linear optical sensor disposed distal of the nozzle and configured to receive the second portion of the beam of light.

In some embodiments, the first linear optical sensor comprises a linear array of sensor elements disposed along a third axis. In some embodiments, the light source comprises a light element and a collimating lens configured to collimate the beam of light along at least one axis. In some embodiments, the system comprises a collimating lens coupled to the linear optical sensor, where the collimating lens is configured to focus the beam of light in a line toward the linear optical sensor. In some embodiments, the collimating lens is disposed adjacent to the linear optical sensor. In some embodiments, the system comprises a second linear optical sensor disposed adjacent to the first linear optical sensor, where the first and second linear optical sensors are directed in a fourth axis toward the light source. In some embodiments, the fourth axis is parallel to the second axis.

In some embodiments, the system comprises a processor configured to: receive, from the first linear optical sensor, a first plurality of displacement measurements obtained over a first period of time; determine, based on the first plurality of displacement measurements and the first period of time, an intraocular pressure of the eye. In some embodiments, the processor is further configured to: receive, from the first linear optical sensor, a second plurality of displacement measurement obtained over a second period of time preceding the first period of time; detect, based on the second plurality of displacement measurements and the second period of time, a blink; and cause the linear optical sensor to obtain, based on detecting the blink, the first plurality of displacement measurements.

In some embodiments, the system comprises a drug delivery module positioned and oriented to eject a stream of a pharmaceutical agent into the eye, wherein the processor is further configured to: cause the drug delivery module to eject the stream of the pharmaceutical agent into the eye; receive, from the linear optical sensor, a third plurality of displacement measurement obtained over a third period of time; determine, based on the third plurality of displacement measurements whether the stream of the pharmaceutical agent reached the eye.

In some embodiments, the system comprises a third optical sensor directed toward the first axis, wherein the processor is configured to determine, based on a proximity measurement from the third optical sensor, whether the eye is disposed within measurement range of the nozzle. In some embodiments, the first linear optical sensor comprises a one-dimensional array of photodiodes. In some embodiments, the first linear optical sensor comprises a two-dimensional array of photodiodes. In some embodiments, the light source is configured to emit light having a center wavelength in the visible spectrum.

In another embodiment of the present disclosure, a method for measuring intraocular pressure (IOP) of a patient's eye is provided. The method may include generating a puff of air directed in a first axis toward a cornea of the patient's eye, wherein the puff of air comprises a pressure. The method may further include emitting, by a light source, a beam of light toward a first optical sensor array along a second axis transverse to the first axis, wherein the beam of light comprises a width in the first axis such that a first portion of the beam of light illuminates a lateral surface of the eye and a second portion of the beam of light passes in front of the eye. The method may further include detecting, by the first optical sensor array, a width of the second portion of the beam of light. The method may further include determining, based on the detected width of the second portion of the beam of light and the pressure of the puff of air, the IOP of the patient's eye.

In some embodiments, detecting the width of the second portion of the beam of light comprises: receiving, from the first optical sensor array, a first plurality of displacement measurements over a period of time. In some aspects, the determining the IOP is based on the first plurality of displacement measurements. In some embodiments, determining the IOP comprises: determining an applanation of the cornea. In some embodiments, the determining the IOP is based on a timing of the applanation of the cornea. In some embodiments, determining the timing of the applanation of the cornea comprises determining a maximum displacement based on the first plurality of displacement measurements.

In some embodiments, the method further comprises: receiving, from the first optical sensor array, a second plurality of displacement measurement obtained over a first period of time; detecting, based on the second plurality of displacement measurements, a blink; and causing the first optical sensor array to obtain, based on detecting the blink, the first plurality of displacement measurements at a second time subsequent to the first period of time. In some embodiments, the method further comprises detecting, by a pressure sensor, the pressure of the puff of air.

This summary is provided to introduce a selection of concepts in a simplified form that are further described below in the detailed description. This summary is not intended to identify key features or essential features of the claimed subject matter, nor is it intended to limit the scope of the claimed subject matter. A more extensive presentation of features, details, utilities, and advantages of the tonometry devices and methods of the present disclosure is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a perspective view of a non-contact tonometer, according to at least one embodiment of the present disclosure.

FIG. 2 is a diagrammatic view of a non-contact tonometer having a transverse, array-based optical corneal profile measurement subsystem, according to at least one embodiment of the present disclosure.

FIG. 3 is a top elevation view of a non-contact tonometer having a transverse, array-based optical corneal profile measurement subsystem, according to at least one embodiment of the present disclosure.

FIG. 4 is a front elevation view of a non-contact tonometer having a nozzle, proximity sensor, and a transverse, array-based optical corneal profile measurement subsystem, according to at least one embodiment of the present disclosure.

FIG. 5 is a diagrammatic view of a non-contact tonometer having a transverse, array-based optical corneal profile measurement subsystem, according to at least one embodiment of the present disclosure.

FIG. 6 is a diagrammatic view of a non-contact tonometer having a transverse, array-based optical corneal profile measurement subsystem, according to at least one embodiment of the present disclosure.

FIG. 7 is a diagrammatic view of a non-contact tonometer having a transverse, array-based optical corneal profile measurement subsystem, according to at least one embodiment of the present disclosure.

FIG. 8 is a front elevation view of a non-contact tonometer having a nozzle, proximity sensor, and a transverse, array-based optical corneal profile measurement subsystem, according to at least one embodiment of the present disclosure.

FIG. 9 is a graph depicting the displacement-vs.-time relationship of a cornea deflecting in response to a jet of air from a non-contact tonometry device in accordance with at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while devices of the present disclosure are described in terms of portable devices configured to direct air toward a human eye, it is understood that the disclosure is not intended to be limited to this application. The devices and systems are equally well suited to any application requiring pumping of brief puffs, pulses, or jets of air with certain profiles of pressure, density, and flow rate. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Presently, treatment of glaucoma mainly consists of periodically administering injections or eye drops to reduce intraocular pressure (IOP). However, the effectiveness of pharmaceuticals can greatly vary from patient-to-patient. Furthermore, the IOP for a given patient can vary significantly based on time of day, exercise, medication use, and other factors. This means that any given measurement is subject to considerable uncertainty, so it may take a plurality of measurements over time to provide confidence as to the health status of the patient and the proper dosage of medications. Effective treatment of glaucoma requires adherence to dosage schedules, and an accurate knowledge of the patient's IOP. The more recent the measurement is, the more it will reflect the patient's current condition, and thus the more effective the treatment will be. IOP measurements in a doctor's office, taking place once or twice per year, are unable to account for variation in patient IOP and measurements may grow stale due to time lag since the most recent measurement.

IOP may be measured through noncontact tonometry, which involves directing a puff of air at the patient's eye and measuring the resulting deflection. The air pressure required to temporarily flatten a region of the patient's cornea is equal to the patient's IOP. A plurality of measurements of the cornea are obtained by an optical sensor configured to receive light reflected from the cornea. The light source and light sensor may be oriented at oblique angles relative to the central axis of the eye such that the axis of the light source and the axis of the light sensor intersect at a point or location on or near the surface of the cornea. In some aspects, this arrangement of the light source and light sensor receiving reflected rays from the cornea may be somewhat inflexible, in that accurate measurements depend on precise placement of the cornea in all three axes. For example, if the patient's cornea is not positioned precisely at or near the point of intersection of the light source axis and the light sensor axis, it may be unlikely to obtain an accurate IOP measurement.

The present disclosure describes devices, systems, and methods for obtaining measurements of a cornea for non-contact tonometry. Embodiments of the present disclosure include non-contact tonometers having an air puff generator, a light source, and an optical sensor array. In some aspects, the light source and optical sensor array may be positioned with respect to one another such that the sensor is configured to receive at least a portion of the light rays directly from the light source. In other words, the optical sensor array may be positioned with respect to the light source such that the optical sensor array is in a direct optical path of the light source. In an example embodiment, the light source and optical sensor array are positioned and oriented transversely to the optical axis of the eye. Accordingly, the light source may project a beam of light on at least a portion of a side of the eye where the width of the beam spans at least a portion of the depth of the cornea and extends beyond the cornea. The cornea may prevent or inhibit at least a portion of the light rays from reaching the sensor array, while a portion of the light rays pass in front of the cornea to the optical sensor array. The sensor array may obtain several measurements within a period of time corresponding to the air puff. For example, the linear sensor may be configured to obtain 10, 50, 100, 200, 500, 1000, 2000, 5000, and/or any other suitable number of measurements per second. Based on the measurements, the processor may be configured to determine the deflection of the cornea in response to the air puff. Based on the deflection, the processor may be configured to determine or infer the IOP of the eye.

Embodiments of the present disclosure may include optical components to create collimated light beams, diverging light beams, fanned light beams, and/or any other suitable shape of beam. The beam may have a width of several millimeters (e.g., 5 mm, 10 mm, 15 mm, 20 mm, 25 mm, 50 mm, etc.), where the width is in the z-axis, or optical axis of the eye. The width of the beam may allow for more flexible positioning and/or misalignment of the cornea during the measurement. For example, the width of the beam may allow for the cornea to be positioned, in the z-axis, within a range of several millimeters. In some embodiments, a tonometry device may further include one or more focusing optics to focus the light rays having passed by the cornea in at least one of the y-axis or the z-axis. In some aspects, the focusing optics may allow for flexibility in the positioning of the cornea and one of the x-axis or the y-axis. For example, the focusing optics may include a rod lens configured to focus a beam having a beam height onto a line or more focused position in the y-axis corresponding to the sensor array. For example, the focusing optics may be configured to focus the beam into a line that is parallel with the sensor array. Accordingly, the eye may be positioned in a range of locations in both the z-axis and the y-axis while still providing an accurate IOP measurement. In some aspects, by using a beam that is collimated in at least the z-axis, the measurements may also be less sensitive to positioning in the x-axis. Accordingly, the various arrangements described herein may provide a more robust and user-friendly tonometry system, which may be suitable for home use without the supervision of a physician. In another aspect, the frequencies or wavelengths of the light used in the corneal profile measurement may include infrared, visible light, and/or any other suitable frequency. In this regard, because the light rays from the light source can be oriented transverse to the optical axis of the eye, higher energy (e.g., higher frequency) light may be used with relatively little risk of ocular damage.

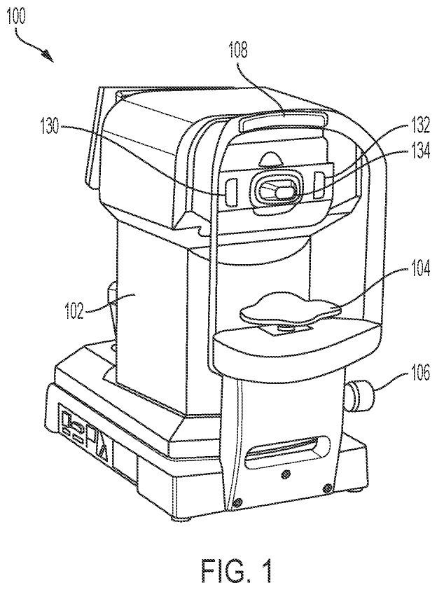

FIG. 1 is a perspective view of a tonometer 100. The tonometer 100 includes a housing 102 enclosing and/or supporting a plurality of components. The tonometer 100 further includes a chin rest 104, an adjustment dial 106, and a head rest 108. The chin rest 104 and head rest 108 provide stopping features for more consistent and repeatable placement of the user's eye near an eye cup 134. The adjustment dial 106 may be configured to make adjustments in one or more axes to position the user's eye within an acceptable range of positions near the eye cup 134. In some aspects, the adjustment dial 106 may also be used to switch from a left eye position to a right eye position, and vice versa.

The tonometer 100 further includes a light source 130 and a sensor array 132. The light source 130 is positioned and oriented to project a beam of light across the eye in a direction transverse to the optical axis of the eye. Similarly, the sensor array 132 is positioned to collect at least a portion of the light from the light source 130, where the collected light travels transversely to the optical axis of the eye. In some aspects, the sensor array 132 is configured to receive the portion of the light that is not obscured by the user's cornea. When a jet or puff of air is delivered through the eye cup 134, the cornea may flatten or compress. Accordingly, a greater amount of the light from the light source 130 may reach the sensor array 132 in the moments in time when the cornea is deflected or compressed to let more light pass through. In some aspects, the sensor array includes a plurality of sensor elements positioned along the z-axis or optical axis. The z-axis may be described as being parallel to the direction in which the air puff is delivered, and/or parallel to the optical axis of the eye. Accordingly, the number of sensors registering at least a threshold amount of light in the array may correspond to the deflection of the cornea. Thus, the deflection or compression of the cornea due to the air puff over the period of time in which the eye receives the air puff may be determined based on the readings from the sensor array 132.

The light source 130 and the sensor array 132 may be controlled by a processor or controller contained within the housing 102. The light source 130 and the sensor array 132 may be controlled together with the air puff generator components (e.g., pump, valve, etc.) such that the light source 130 and the sensor array 132 are only activated in a time period in which the air puff will impinge on the cornea. The light source 130 may be configured to create a collimated beam having a width between 5 mm and 30 mm wide in at least the z-axis. In some embodiments, the collimated beam is between 10 mm and 20 mm wide in the z-axis. In some embodiments, the collimated beam is cylindrical. In other embodiments, the collimated beam is oblong, flat, converging, diverging, or any other suitable profile. In some embodiments, the light source 130 is configured to output a pulsed beam. In other embodiments, the light source 130 is configured to output a continuous beam.

The tonometer 100 may be a benchtop or desktop tonometer device. In some embodiments, the tonometer 100 is configured for home use such that the tonometer 100 can be used by the patient without supervision or assistance by a physician. The tonometer 100 may include one or more user controls for controlling the IOP measurement procedure. The tonometer 100 may also include a user interface device for displaying instructions, providing feedback for the user or appropriate positioning of the eye along the air puff axis, displaying the IOP measurements, and/or any other suitable interface function. Tonometers configured for home use may be desirable and are advantageous in some aspects. In this regard, accurate, frequent, and more immediate IOP measurements may allow for a more tailored or individual treatment regimen. In this regard, if the patient obtains more measurements throughout the day at various times, the patient may obtain more information about the patient's variations in IOP throughout the day. Accordingly, the physician and the patient may create a treatment schedule or regimen that more precisely fits the patient's indications. In other embodiments, the tonometer 100 may be configured for use by a physician, for example, in an ophthalmologist's or optometrist's office. In other embodiments, the tonometer 100 may be a mobile or portable device. For example, the tonometer 100 may be configured for handheld operation. For example, the tonometer 100 may include a rechargeable battery so that the tonometer 100 can be used without being plugged in to a power outlet.

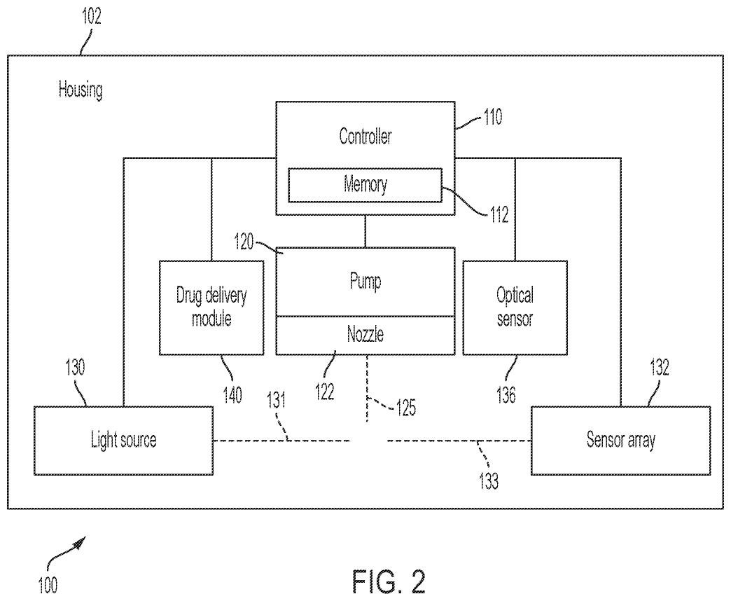

FIG. 2 is a diagrammatic view of a tonometer 100. The tonometer 100 shown in FIG. 2 may be similar in some aspects to the tonometer 100 shown in FIG. 1. In this regard, the tonometer 100 shown in FIG. 2 includes a housing 102, a light source 130, and a sensor array 132. The light source 130 is configured to emit a beam of light along a first axis 131. The sensor array 132 is configured to receive light along at least a second axis 133. The tonometer 100 further includes a pump 120 and a nozzle 122 configured to generate an air puff or air jet. The air puff travels along an axis 125. The axis 125 may be aligned with, or substantially aligned with, an optical axis of the eye. Further, the axis 125 may be orthogonal, perpendicular, or otherwise transverse to one or both of the first axis 131 and/or the second axis 133.

The tonometer 100 further includes a drug delivery module 140 configured to deliver or administer a pharmaceutical agent to the patient's eye. For example, the drug delivery module 140 may be configured to produce a stream or mist of an ophthalmic fluid to the eye as part of a treatment regimen. In some aspects, the delivery of the pharmaceutical agent by the module 140 may be based on the IOP measurements obtained by the tonometer 100. The tonometer 100 further includes an optical sensor 136 different from the sensor array 132. The optical sensor 136 may be configured for proximity measurements, in some embodiments. For example, the optical sensor 136 may be configured to determine whether the eye is within a suitable range of the nozzle 122. In some aspects, the optical sensor 136 may be controlled by the controller 110 for determining whether there is a user at the tonometer to activate the components of the tonometer 100. In some aspects, the optical sensor 136 may be used for blink detection. In this regard, the controller 110 may be configured to detect a blink based on optical measurements from the optical sensor 136, and to activate the pump 120 and/or nozzle 122 after detecting the blink. In some aspects, the pump 120 includes a valve, and the controller 110 may be configured to activate the valve to release the puff of air. In other embodiments, the controller 110 may detect blinks using the light source 130 and the optical sensor array 132. For example, the controller 110 may detect a momentary increase in the corneal profile based on signals from the optical sensor array, and determine that a blink has occurred.

In some aspects, the pressure exerted by the air puff on the cornea increases over a brief period of time (in an example, 15 milliseconds), until it is sufficient to cause temporary applanation or flattening of the cornea, and then a brief period of slight concavity. The pressure may then decrease over a period of time (e.g., 15 ms) such that the cornea flattens again before returning to its normal shape. In both moments of applanation, a noncontact tonometer detects the applanation with an optical sensor.

In an example, the static pressure of the air puff on the center of the cornea reaches about 30 mmHg (4.0 kPa or 0.04 atmospheres) above ambient pressure, with an accuracy of about ±1 mmHg, or less than ±1 mmHg. For example, the accuracy of the pressure of the air puff created by the pump 120 and the nozzle 122 may be ±0.05 mmHg, ±0.1 mmHg, ±0.5 mmHg, or any other suitable accuracy. Assuming the patient's IOP is somewhere between 5 mmHg and 30 mmHg, the air puff may result in two separate applanation events—one during the rise time and one during the fall time. In some embodiments, the tonometer 100 may further include one or more pressure sensors within the pump 120, the nozzle 122, and/or at any other suitable position to monitor the pressure of the air puff expelled through the nozzle 122. The controller 110 receiving these measurements then has two separate IOP readings that may be reported separately, averaged, or otherwise.

The tonometer 100 includes the controller 110 and a memory 112 in communication with the controller 110. The memory 112 may store instructions executable by the memory 112 for performing one or more of the functions described above. In some embodiments, the memory 112 may further store IOP measurements, patient-related data, and/or any other suitable type of information. In some embodiments, the memory 112 may store treatment-related data for generating treatment alerts or indicators for the patient. For example, the memory 112 may store general and/or patient-specific IOP thresholds for determining whether to output an alert to the user or a network, or to cause the drug delivery module 140 to deliver a pharmaceutical agent to the patient's eye.

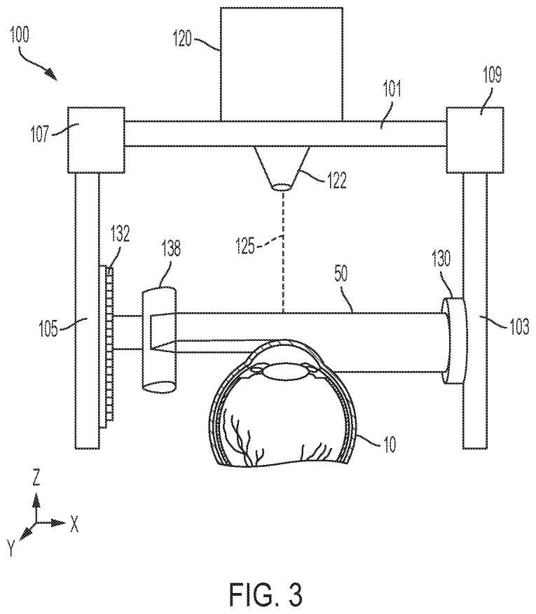

FIG. 3 is a top elevation view of a non-contact tonometer having a transverse, array-based optical corneal profile measurement subsystem, according to at least one embodiment of the present disclosure. The tonometer 100 includes a pump 120, a nozzle 122 coupled to a first housing portion 101. The tonometer 100 further includes a light source 130 coupled to a second housing portion 103, and a sensor array 132 coupled to a third housing portion. The first housing portion 101 is coupled to the second housing portion 103 by a first connector element 109, and the first housing portion 101 is coupled to the third housing portion 105 by a second connector element 107. In some embodiments, the connector elements 107, 109 are adjustable to adjust the positions of the light source 130, the sensor array 132, and/or the focusing element 138. For example, in some embodiments, the connector elements 107, 109 may include telescoping features, sliding clamps, rack-and-pinion mechanisms, hinges, and/or any other suitable adjustment mechanism.

The pump 120 and nozzle 122 are configured to produce a puff or jet of air along an axis 125. The puff of air may have a controlled profile, pressure, duration, speed, and/or any other pneumatic characteristic. The light source 130 is configured to create a beam 50 of light in a direction that is transverse or perpendicular to the axis 125. It will be understood that the beam 50 may not be exactly perpendicular to the axis 125, in some embodiments. For example, the beam 50 may be centered along an axis or direction that is between 75°-105° with respect to the axis 125. In FIG. 3, the beam 50 is collimated. For example, in some embodiments, the light source 130 may include a laser element. In some embodiments, the laser element may be an infrared (IR) laser element. In other embodiments, the laser element may be a visible light laser element.

The beam 50 comprises a width or thickness in at least the direction of the first axis 125. Accordingly, the beam 50 impinges the eye 10 at a range of depths that includes the cornea. A first portion of the beam 50 is obscured by the eye, and a second portion of the beam 50 that is not obscured by the eye 10 continues forward to the focusing element 138. The focusing element 138 may include a rod lens, in some embodiments. In other embodiments, other types of focusing elements may be used, including Fresnel lenses, focusing mirrors, and/or any other suitable type of focusing elements. The focusing element 138 is configured to focus the beam 50 in at least one axis. For example, in FIG. 3, the focusing element 138 is configured to focus the beam 50 in the y-axis, but not in the z-axis. In this respect, the focusing element 138 being positioned proximate the sensor array 132 may allow for some misalignment of the eye in the y-axis. For example, because the beam 50 has a width in the y-axis, there may be a range of suitable positions in the y-axis at which the sensor array 132 can obtain accurate measurements of the corneal profile and deflection.

The sensor array 132 includes a plurality of sensor elements arranged along the z-axis (parallel to the axis 125). In the illustrated embodiment, the sensor array 132 is a one-dimensional, linear sensor array. Each element of the array 132 may have a width in the z-axis, and a spacing in the z-axis. The width and spacings of the elements of the array 132 may be sufficiently small to detect a range of deflections indicating a range of IOPs. For example, each element of the array 132 may have a sensor width ranging between 2 um to 50 um and an inter-element spacing ranging between 3 um and 60 um. The elements may also have a height in the y-axis. In some embodiments, the y-axis height of the elements of the array 132 may provide additional flexibility in the corneal position relative to the array 132 and beam 50. The array 132 may include any suitable number of elements, including 20, 48, 64, 128, 256, 512, 1024, 2048 and/or any other suitable number of elements, both greater or smaller. In some embodiments, the elements of the array 132 include photodiodes configured to convert received light into an electrical voltage. The elements of the array 132 may be coupled to one another by a bus. In some embodiments, the array 132 includes a multiplexer to multiplex the signals from each of the electrical elements and transmit the signals to a controller (e.g., controller 110, FIG. 1). In one embodiment, the array 132 includes a Hamamatsu S11639-01 (which has 2048 pixels that measure 14 um in width and a total sensor width of 28.672 mm.

In some embodiments, the sensor array 132 may be configured to provide a signal indicating a voltage and/or current for each element of the array 132. In other embodiments, the sensor array 132 may be configured to provide a signal indicating a binary value, for each element, representing whether the sensor element received light above a threshold amount. Accordingly, the signal provided by the array 132 may indicate a number of sensor elements that are illuminated by the light source 130, which is based on the flattening or deflection of the cornea. Based on the timing of the flattening of the cornea and the known pressure and characteristics of the air puff, the tonometer 100 can determine the IOP. In this regard, because the beam of light 50 impinges on the cornea from the side, the tonometer 100 may determine the time between the air puff reaching the cornea, and the lateral measurement of the cornea first reaching a minimum.

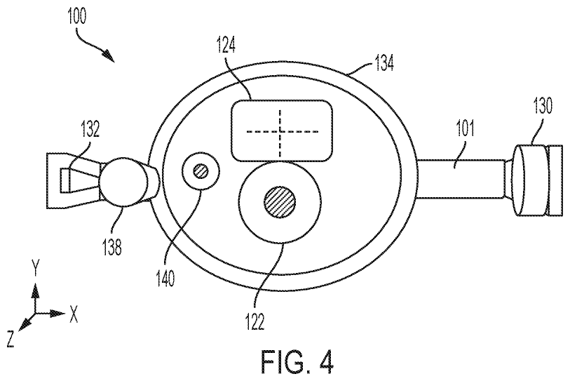

FIG. 4 is a front elevation view of the non-contact tonometer 100 shown in FIG. 3, according to embodiments of the present disclosure. The tonometer 100 includes the light source 130 positioned opposite, and configured to project a beam of light towards the sensor array 132. The focusing element 138 comprises a cylindrical rod lens for focusing beams of light from the light source 130 onto the sensor array 132. The tonometer 100 further includes a housing portion 134, to which the nozzle 122, a drug delivery module 140, and user alignment feature 124 are coupled. The housing portion 134 may comprise an eye cup, in some embodiments. However, the eye cup may be positioned so as not to prevent passage of a beam of light between the light source 130 and the sensor array 132. The drug delivery module 140 may include a dispensing nozzle for producing a stream or mist of a pharmaceutical agent. The user alignment feature 124 may include a display, screen, mirror, and/or any other suitable feature for guiding the user to position the cornea in an acceptable position for performing an IOP measurement. For example, the user alignment feature 124 may include a set of cross hairs, circles, or any other suitable type of shape, where the overlapping of the shapes indicates that the user's eye is correctly positioned in at least one of the axes.

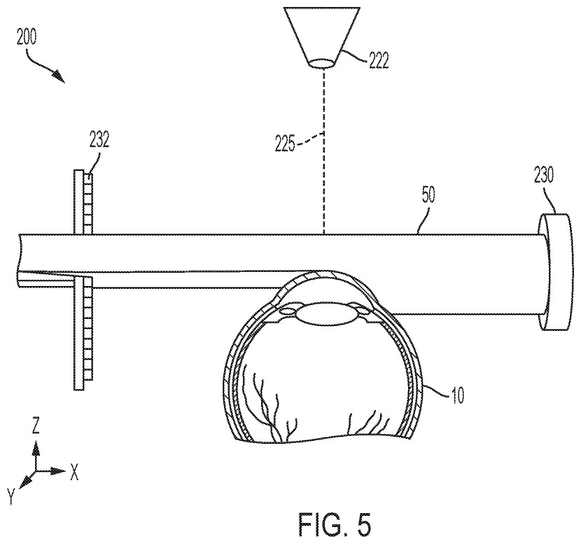

FIG. 5 is a diagrammatic view of a non-contact tonometer having a transverse, array-based optical corneal profile measurement subsystem, according to another embodiment of the present disclosure. Similar to the tonometer 100 shown in FIG. 3, the tonometer 200 shown in FIG. 5 includes a nozzle 222, a light source 230, and a sensor array 232. The nozzle 222, light source 230, and sensor array 232 may be coupled to respective housing portions, similar to the embodiment shown in FIG. 3. Further, the positions and/or orientations of the light source 230 and sensor array 232 may be adjustable by adjustable connection components of the housing, as similarly described above. In the embodiment of FIG. 5, no focusing element is present.

The nozzle 222 may be configured to produce a puff or jet of air along an axis 225. The puff of air may have a controlled profile, pressure, duration, speed, and/or any other pneumatic characteristic. The light source 230 is configured to create a beam 50 of light in a direction that is transverse or perpendicular to the axis 225. It will be understood that the beam 50 may not be exactly perpendicular to the axis 225, in some embodiments. For example, the beam 50 may be centered along an axis or direction that is between 75°-105° with respect to the axis 225. In FIG. 3, the beam 50 is collimated. For example, in some embodiments, the light source 230 may include a laser element. In some embodiments, the laser element may be an infrared (IR) laser element. In other embodiments, the laser element may be a visible light laser element.

The beam 50 comprises a width or thickness in at least the direction of the first axis 225. Accordingly, the beam 50 impinges the eye 20 at a range of depths that includes the cornea. A portion of the beam 50 that is not obscured by the eye 20 continues forward to the sensor array 232. In some aspects, the lack of the focusing element in the embodiment of FIG. 5 may allow for less error or misalignment in eye position than the embodiment shown in FIG. 3, for example.

As similarly described above with respect to FIG. 3, the sensor array 232 includes a plurality of sensor elements arranged along the z-axis (parallel to the axis 225). In the illustrated embodiment, the sensor array 232 is a one-dimensional, linear sensor array. Each element of the array 232 may have a width in the z-axis, and a spacing in the z-axis. The width and spacings of the elements of the array 232 may be sufficiently small to detect a range of deflections indicating a range of IOPs. For example, each element of the array 232 may have a sensor width ranging between 2 um and 50 um and an inter-element spacing ranging between 3 um and 60 um. The array 232 may include any suitable number of elements, including 20, 48, 64, 228, 256, 512, 1024, 2048, and/or any other suitable number of elements, both greater or smaller. In some embodiments, the sensor array 232 may include multiple rows of sensor elements stacked in the y-axis. For example, the sensor array 232 may comprise a two-dimensional array. The tonometer 200 may comprise a controller configured to detect, based on signals from the two-dimensional array, the position of the cornea in the y-axis. Based on the determined position in the y-axis, the controller may further determine the amount of deflection and/or the timing of the deflection of the cornea.

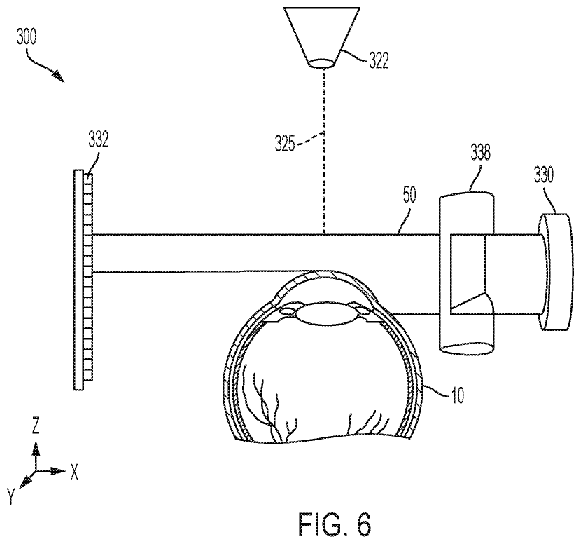

FIG. 6 is a diagrammatic view of a non-contact tonometer having a transverse, array-based optical corneal profile measurement subsystem, according to another embodiment of the present disclosure. Similar to the tonometer 100 shown in FIG. 3, the tonometer 300 shown in FIG. 6 includes a nozzle 322, a light source 330, a sensor array 332, and a focusing element 338. The nozzle 322, light source 330, sensor array 332, and focusing element 338 may be coupled to respective housing portions, similar to the embodiment shown in FIG. 3. Further, the positions and/or orientations of the light source 330 and sensor array 332 may be adjustable by adjustable connection components of the housing, as similarly described above. In the embodiment of FIG. 6, the focusing element 338 is positioned proximate to the light source 330, instead of proximate to the sensor array 332.

The nozzle 322 may be configured to produce a puff or jet of air along an axis 325. The puff of air may have a controlled profile, pressure, duration, speed, and/or any other pneumatic characteristic. The light source 330 is configured to create a beam 50 of light in a direction that is transverse or perpendicular to the axis 325. It will be understood that the beam 50 may not be exactly perpendicular to the axis 325, in some embodiments. For example, the beam 50 may be centered along an axis or direction that is between 75°-105° with respect to the axis 325. In FIG. 6, the beam 50 is initially collimated, and is then focused to a narrow line by the focusing element 338. The focusing element 338 is positioned proximate to the light source 330. The focusing element 338 may comprise a rod lens, in some embodiments. In other embodiments, the focusing element 338 may comprise a semi-cylindrical lens

The focused beam 50 comprises a width or thickness in the direction of the first axis 325. Accordingly, the beam 50 impinges the eye 10 at a range of depths that includes the cornea. A portion of the beam 50 that is not obscured by the eye 10 continues forward to the sensor array 332. In some aspects, the beam 50 may have a focal point at or near the sensor array 332. Accordingly, the beam 50 may be converging between the focusing element 338 and the sensor array 332.

As similarly described above with respect to FIG. 3, the sensor array 332 includes a plurality of sensor elements arranged along the z-axis (parallel to the axis 325). In the illustrated embodiment, the sensor array 332 is a one-dimensional, linear sensor array. Each element of the array 332 may have a width in the z-axis, and a spacing in the z-axis. The width and spacings of the elements of the array 332 may be sufficiently small to detect a range of deflections indicating a range of IOPs. For example, each element of the array 332 may have a sensor width ranging between 2 um to 50 um and an inter-element spacing ranging between 3 um to 60 um. The array 332 may include any suitable number of elements, including 20, 48, 64, 328, 256, 512, 1024, 2048, and/or any other suitable number of elements, both greater or smaller.

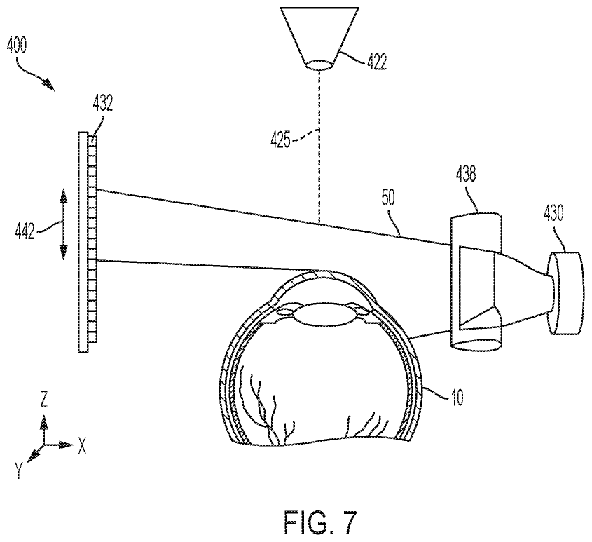

FIG. 7 is a diagrammatic view of a non-contact tonometer having a transverse, array-based optical corneal profile measurement subsystem, according to another embodiment of the present disclosure. Similar to the tonometer 100 shown in FIG. 6, the tonometer 400 shown in FIG. 7 includes a nozzle 422, a light source 430, a sensor array 432, and a focusing element 438. The nozzle 422, light source 430, sensor array 432, and focusing element 438 may be coupled to respective housing portions, similar to the embodiment shown in FIG. 3. Further, the positions and/or orientations of the light source 430 and sensor array 432 may be adjustable by adjustable connection components of the housing, as similarly described above.

In the embodiment of FIG. 7, the beam 50 produced by the light source 430 is a diverging beam. In some aspects, the light source 430 is configured to emit light as a diverging beam. In other embodiments, the light source 430 includes one or more optical components (e.g., lens, mirror) configured to create the diverging beam 50 based on light emitted by the light source 430. The tonometer 400 includes a focusing element 438 proximate to the light source 430. The focusing element 438 is configured to focus the diverging beam 50 in the y-axis, creating a fanned beam that diverges in the z-axis and converges in the y-axis. Accordingly, the focused beam 50 comprises a width or thickness in the direction of the first axis 425 that grows as the beam 50 approaches the sensor array 432. The beam 50 impinges the eye 10 at a range of depths that includes the cornea. In some aspects, the diverging nature of the beam 50 may magnify the variation in the amount of light that reaches the sensor array 432 due to the compressed cornea from the air puff. For example, if the cornea compresses 4 mm, the width 442 of the portion of the beam 50 received at the sensor array 432 may change by 4.5 mm, for example. In another embodiment, if the cornea compresses by 4 mm, the width 442 of the portion of the beam 50 that reaches the sensor array 432 may change by 1.2 mm, 1.4 mm, 1.6 mm, 1.8 mm, 2.0 mm, and/or any other suitable amount. The more prominent change in the width 442 based on the diverging beam may improve the resolution of the cornea deflection measurements, in some aspects. For example, the more prominent change in the width 442 may increase the number of sensor elements that receive rays of the beam 50 due to the compression or deflection of the cornea.

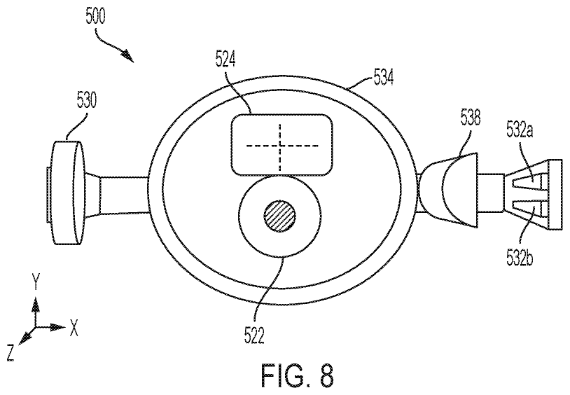

FIG. 8 is a front elevation view of a non-contact tonometer 500, according to embodiments of the present disclosure. The tonometer 500 includes a light source 530 positioned opposite, and configured to project a beam of light towards, the sensor array 532. The tonometer 500 includes a focusing element 538 for focusing beams of light from the light source 530 onto the sensor array 532. The tonometer 500 further includes a housing portion 534, to which a nozzle 522, and a user alignment feature 524 are coupled. The user alignment feature 524 may include a display, screen, mirror, and/or any other suitable feature for guiding the user to position the cornea in an acceptable position for performing an IOP measurement. For example, the user alignment feature 524 may include a set of cross hairs, circles, or any other suitable type of shape, where the overlapping of the shapes indicates that the user's eye is correctly positioned in at least one of the axes.

The focusing element 538 comprises a semi-cylindrical lens configured to focus light from the light source 530 onto one or both of a first sensor array 532a and/or a second sensor array 532b. The sensor arrays 532a, 532b, may be identical, and arranged in a stacked relationship in the y-axis. In other embodiments, the sensor arrays 532a, 532b may be different sensor arrays. In other embodiments, more than two sensor arrays 532 may be used. For example, the tonometer 500 may include several rows of sensor elements. In some embodiments, a two-dimensional sensor array may be used. Such an arrangement may allow for greater flexibility in the y-axis, since there are multiple rows of sensor elements at different positions in the y-axis to receive the light from the light source 530.

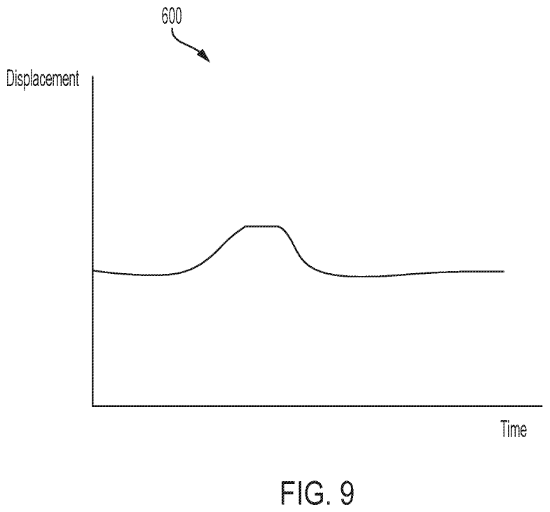

FIG. 9 is a graph of a corneal displacement over time. The corneal displacement is due to the compression of the cornea in response to the air puff from the nozzle of a tonometer. Accordingly, with higher amounts of compression, the graph 600 shows higher amounts of corneal displacement. The displacement reaches a plateau at the point of corneal applanation. At this point, the cornea may become slightly concave. However, because the incident beam approaches the eye laterally, the measured displacement may not detect the momentary concavity of the cornea. The displacement may be measured by a sensor array, such as a linear sensor array. For example, the sensor array may include a plurality of sensor elements corresponding to different depths in the displacement direction. The size and spacing of the sensor elements may be sufficiently small to detect changes in IOP with an accuracy of ±0.05 mmHg, ±0.1 mmHg, ±0.2 mmHg, ±0.5 mmHg, ±1 mmHg, and/or any other suitable accuracy.

In some aspects, a processor (e.g., the controller 110, FIG. 1) is configured to calculate an IOP based on the corneal displacement measurements obtained by the sensor array (e.g., 132, 232, 323, 432, 532a, 532b). In this regard, because the displacement may be associated with the number of sensor elements of the sensor array that are illuminated by the beam of light, the processor may determine the IOP based on the number of sensor elements illuminated by the beam of light. In some aspects, the IOP measurement may be based on known parameters of the air puff, such as the pressure.

The controller could employ any combination of hardware, software, and firmware to perform its functions. The controller could employ a fixed instruction set provided in read-only memory (ROM) or could have an updatable instruction set provided in programmable read-only memory (PROM), electrically erasable programmable read-only memory, flash memory, or any equivalent thereof. Readings taken from pressure sensors may be stored on the device or communicated externally, as may the times the pump is activated, the times the valve is triggered, and/or a running tally of total times the device has been used. The pressure value required to trigger the valve may be adjustable or programmable, as may the speed of the compression pump.

Communication (including but not limited to software updates, firmware updates, or readings from the device) to and from the device 100 could be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. As explained herein, the disclosed pump devices may be included in a non-contact tonometer, such as in a hand-held non-contact tonometer. IOP measurements may be taken using the non-contact tonometer and communicated from the tonometer using the described wireless or wired communication capability.

The logical operations making up the embodiments of the technology described herein may be referred to variously as operations, steps, objects, elements, components, or modules. It should be understood that these may be performed or arranged in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the jet pump for noncontact tonometry. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

The above specification, examples and data provide a complete description of the structure and use of exemplary embodiments of the jet pump for noncontact tonometry as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter. For example, the jet pump could be used to produce controlled puffs of other gases than ambient air, including but not limited to oxygen, nitrogen, helium, and argon, or of gases that contain colorants, odorants, medications, or other materials. Additionally, some or all of the components of the jet pump may be contained within a housing, either alone or with other components such as a battery and/or power supply.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims. Persons skilled in the art will recognize that the devices, systems, and methods described above can be modified in various ways not explicitly described or suggested above. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system for determining an intraocular pressure (IOP) of an eye, the system comprising:
a pump configured to generate a puff of air;
a nozzle in communication with the pump and configured to direct the puff of air along a first axis toward the eye;
a light source disposed distal of the nozzle and directed to emit a beam of light toward the first linear optical sensor along a second axis transverse to the first axis, wherein the beam of light comprises a width in the first axis such that a first portion of the beam of light illuminates a lateral surface of the eye and a second portion of the beam of light passes in front of the eye; and
a first linear optical sensor disposed distal of the nozzle and configured to receive the second portion of the beam of light.

2. The system of claim 1, wherein the first linear optical sensor comprises a linear array of sensor elements disposed along a third axis.

3. The system of claim 1, wherein the light source comprises a light element and a collimating lens configured to collimate the beam of light along at least one axis.

4. The system of claim 1, further comprising a collimating lens coupled to the linear optical sensor, wherein the collimating lens is configured to focus the beam of light in a line toward the linear optical sensor.

5. The system of claim 4, wherein the collimating lens is disposed adjacent to the linear optical sensor.

6. The system of claim 1, further comprising a second linear optical sensor disposed adjacent to the first linear optical sensor, wherein the first and second linear optical sensors are directed in a fourth axis toward the light source.

7. The system of claim 6, wherein the fourth axis is parallel to the second axis.

8. The system of claim 1, further comprising a processor configured to:
receive, from the first linear optical sensor, a first plurality of displacement measurements obtained over a first period of time;
determine, based on the first plurality of displacement measurements and the first period of time, an intraocular pressure of the eye.

9. The system of claim 8, wherein the processor is further configured to:
receive, from the first linear optical sensor, a second plurality of displacement measurement obtained over a second period of time preceding the first period of time;
detect, based on the second plurality of displacement measurements and the second period of time, a blink; and
cause the linear optical sensor to obtain, based on detecting the blink, the first plurality of displacement measurements.

10. The system of claim 8, further comprising a drug delivery module positioned and oriented to eject a stream of a pharmaceutical agent into the eye,
wherein the processor is further configured to:
cause the drug delivery module to eject the stream of the pharmaceutical agent into the eye;
receive, from the linear optical sensor, a third plurality of displacement measurement obtained over a third period of time;
determine, based on the third plurality of displacement measurements whether the stream of the pharmaceutical agent reached the eye.

11. The system of claim 8, further comprising a third optical sensor directed toward the first axis, wherein the processor is configured to determine, based on a proximity measurement from the third optical sensor, whether the eye is disposed within measurement range of the nozzle.

12. The system of claim 1, wherein the first linear optical sensor comprises a one-dimensional array of photodiodes.

13. The system of claim 1, wherein the first linear optical sensor comprises a two-dimensional array of photodiodes.

14. The system of claim 1, wherein the light source is configured to emit light having a center wavelength in the visible spectrum.

15. A method for measuring intraocular pressure (IOP) of a patient's eye, comprising:
generating a puff of air directed in a first axis toward a cornea of the patient's eye, wherein the puff of air comprises a pressure;
emitting, by a light source, a beam of light toward a first optical sensor array along a second axis transverse to the first axis, wherein the beam of light comprises a width in the first axis such that a first portion of the beam of light illuminates a lateral surface of the eye and a second portion of the beam of light passes in front of the eye;
detecting, by the first optical sensor array, a width of the second portion of the beam of light; and
determining, based on the detected width of the second portion of the beam of light and the pressure of the puff of air, the IOP of the patient's eye.

16. The method of claim 15, wherein the detecting the width of the second portion of the beam of light comprises:
receiving, from the first optical sensor array, a first plurality of displacement measurements over a period of time; and
wherein the determining the IOP is based on the first plurality of displacement measurements.

17. The method of claim 16, wherein the determining the IOP comprises:
determining an applanation of the cornea; and
wherein the determining the IOP is based on a timing of the applanation of the cornea.

18. The method of claim 17, wherein the determining the timing of the applanation of the cornea comprises determining a maximum displacement based on the first plurality of displacement measurements.

19. The method of claim 16, further comprising:
receiving, from the first optical sensor array, a second plurality of displacement measurement obtained over a first period of time;
detecting, based on the second plurality of displacement measurements, a blink; and
causing the first optical sensor array to obtain, based on detecting the blink, the first plurality of displacement measurements at a second time subsequent to the first period of time.

20. The method of claim 15, further comprising:
detecting, by a pressure sensor, the pressure of the puff of air.
